(19) United States
(12) Patent Application Publication
(10) Pub. No.: US 2005/0238180 A1
(43) Pub. Date: Oct. 27, 2005

(54) All in one acoustic wireless headphones

(76) Inventor: Jinsuan Chen, Chung Ho City (TW)

Correspondence Address: Donald C. Casey, Suite 100, 311 North Washington Street, Alexandria, VA, 22314, US

(21) Appl. No.: 10/832,317

(22) Filed: Apr. 27, 2004

(51) Int. Cl. A61F 11/06; G10K 11/16; H03B 29/00

(52) U.S. Cl. 381/71.6; 381/74

(57) ABSTRACT

A noise reduction headphone set has been described. The headphone set includes a first circuit system being an anti-noise circuit comprising a microphone for detecting external noise, and means for admitting a wave opposite phase to that of said noise, right and left speakers coupled to said first circuit and variable control means for manually controlling the output of said speakers; an infrared transmitter and a radio circuit system coupled to said transmitter for transmitting an external audio signal switchably coupled to said first circuit whereby signals generated by said first and second circuits cancel external noise, selectively; and power auto/off control circuit coupled to said first and second circuits for shutting down said system in the absence of an audio signal for a predetermined period of time.

### FIELD OF THE INVENTION

This invention relates to wireless headphones and in particular to infrared receiving headphones which includes a noise reduction function.

### DESCRIPTION OF THE PRIOR ART

Wireless headphones have been known for many years, and typically operate with a transmitter receiving audio input from a radio or multimedia source which could be a CD player, television, or the like. The audio output is transmitted to remote speakers or headphones. U.S. Pat. No. RE37884 describes such a system.

In my pending patent application Ser. No. 09/069,947, filed Apr. 30, 1998, there is described a 3-in-1 anti-noise radio sound collection device which consists of a headphone which integrates functions of an anti-noise circuit system, a radio circuit system, and a sound collection circuit system in a single headphone circuit. In that patent application, a microphone was concealed in the headband of the headphones so that the wearer would not have to contend with a separate microphone hardwired into the headphone. The disclosure of patent application Ser. No. 09/069,947, filed Apr. 30, 1998 is hereby incorporated by reference.

In principle, the object of noise reduction is intended to provide an acoustical signal which will cancel out background noise so that the headphone wearer can enjoy audio reception desired without interference from background noise such as an airplane engine, motor vehicle engine, and the like. A signal containing content plus background noise is combined with a signal essential of background noise to cancel the noise from the combined signal.

It is also known that noise reduction can be selective as described in U.S. Pat. No. 5,375,174 with either a radio frequency or infrared system used for communication, which describes noise reduction within a confined area such as an emergency vehicle. This invention is used for emergency personnel to cancel out siren noise. Wireless infrared headphones are also disclosed in pending applications 20030,022,703, and 20010,046,304 as well as in U.S. Pat. Nos. 6,373,942 and 6,035,047.

There remains a need however for an infrared detection system which at the option of the wearer can either function to reduce noise completely, or to reduce noise selectively to permit the user to listen to a desired content signal while screening out background noise. Such a system should be switchable between an infrared source, a cable source and a total silence mode.

### SUMMARY OF THE INVENTION

It has been discovered that a noise reduction headphone set can be provided wherein selective noise reduction is at the option of the wearer and the headphones can receive an infrared signal, or a cable signal also at the option of the user. The headphones of this invention also include a signal detector mechanism wherein the headphone transmitter will automatically shut down when a signal has not been received for a period of time. In this way, then, batteries can be conserved for example if the user puts the headphones down without turning them off.

The device of this invention is intended to include a manually operated switch so that the user can select between complete noise reduction (silence) or selective noise reduction. In either event, the external signal is received, and a compensating noise reduction signal generated to cancel out the unwanted background noise. An audio transmitter receives the infrared input and transmits the same through a radio frequency amplifier, filter and detector. The amount of noise reduction can also be controlled manually by the user.

As noted above, the audio input can either come from an infrared transmitter or from a cable source.

Accordingly it is an object of this invention to provide a portable headphone set wherein an incoming infrared signal to the headphones can, at the option of the user, be canceled, or selectively altered to eliminate unwanted background noise.

It is another object of this invention to provide a noise cancellation headphone set, which operates on an infrared signal, received wirelessly, or on a hardwired cable connection.

It is still another object of this invention to provide a portable noise cancellation headphone set which, when the signal is generated by an infrared transmitter, will detect when the transmitter is no longer generating and shut down the system to conserve battery power.

It is still a further object of this invention to provide a portable noise cancellation headphone set in which the functions are integrated using an infrared input or, selectively a cable input.

These and other objects will become readily apparent with reference to the drawings and following description wherein:

### BRIEF DESCRIPTION OF THE DRAWINGS

FIG. 1 is a block diagram of the wireless infrared noise cancellation headphone set of this invention.

FIG. 2 is a schematic of the wireless noise cancellation headphone set of FIG. 1.

FIG. 3 is a circuit diagram for the headphone set of FIGS. 1 and 2.

### DETAILED DESCRIPTION OF THE INVENTION

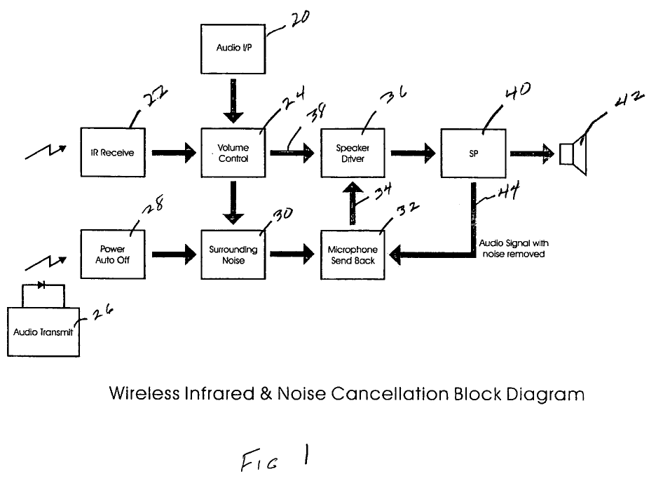

With reference to FIG. 1, audio input and input from an infrared receiver 22 are received by a volume control 24. Volume control 24 typically is a variable resistor controlled by an external dial, which regulates the amount of background noise eliminated. An audio transmitter 26 transmits through a power control 28 to an accumulator 30. The accumulator 30 drives a microphone sendback 32, which has an output 34 to the speaker/driver 36. The output 38 from the volume control also is inputted to the speaker/driver 36. The resulting signal goes to a speaker 40 and to an external speaker 42. The output from the speaker 44 is sent back to the speaker/driver 36 ultimately to the speaker 40.

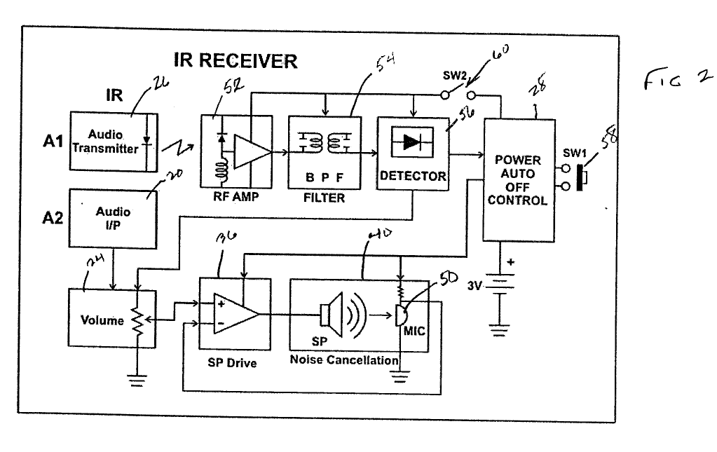

FIG. 2 is a schematic of the IR receiver 22. The receiver 22 contains an audio transmitter denoted in the drawing as A1 and an audio input 20 identified as A2. The signal from the volume control 24 is transmitted to the speaker/driver 36, and to the speaker 40, which contains a noise cancellation function through microphone 50. The signal from transmitter 26 is directed to an amplifier 52, through a filter 54 and a detector 56. The signal is transmitted through the power on/off control 28 and detected therein. In the event a transmitted signal is not detected by the control 28 for a predetermined period of time, the control 28 will shut down the system. In this way the batteries will be maintained in a charged status as long as possible.

In FIG. 2 there is shown a switch Sw1 (58) and a switch Sw2 (60). In this figure the switches are both shown closed. If the wireless and noise reduction functions are both to be on at the same time, both switches 58 and 60 are closed completing the circuits. In the event the user wishes to have the noise reduction only, for total quiet switch 1 is closed and switch 2 remains open. This latter function would be, for example, if the user is on an airplane and desires to sleep without background noise interference.

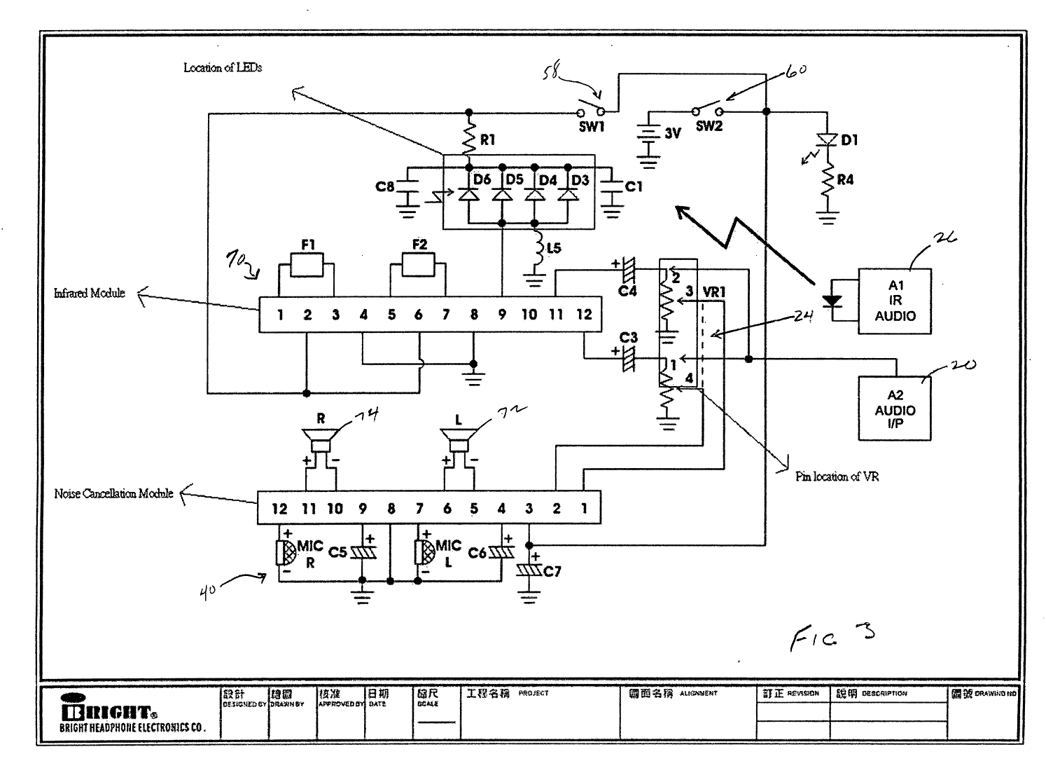

FIG. 3 is a circuit diagram for the receiver of FIG. 2 and shows input to the infrared module 70 from either infrared audio A1 reference No. 26 or infrared audio A2 reference 20. The latter input is through the volume control 24 wherein the variable resistor VR1 is used to control input through the infrared module 70. The circuit also provides input into the noise reduction module 40 wherein left and right microphones (MIC) are used with a left and right speakers 72 and 74 to provide the noise reduction functions.

In summary, the instant invention includes a noise reduction function integrally coupled to an infrared speaker system for headphones. In this way the infrared input can have a selected amount of background noise removed or can be entirely squelched so that the user hears only silence.

As is well known, typically an input signal carries concurrent plus background noise. If the background noise is separately received without the concurrent it can be combined with the concurrent signal in and out of phase relationship and thereby cancel the background noise. By using a variable resistor control, some, a portion, or all of the background noise can be thereby eliminated. Typically infrared signals are used in a confined air space, but in the instance of this invention, a more broad application is provided.

Furthermore, the modular functions are integrally contained within a system compact enough to be mounted in headphones so that the user has only a minimal control, and a dial-up ability to affect that control.

It will be readily seen by one of ordinary skill in the art that the present invention fulfills all of the objects set forth above. After reading the foregoing specifications, one of ordinary skill will be able to effect various changes, substitutions or equivalents and various other aspects of the invention as broadly disclosed herein. It is therefore intended that the protection granted hereon be limited only by the definition contained in the appended claims and equivalents thereof.

1. A wireless infrared signal producing headphone device with anti-noise headphone device for transmitting either an anti-noise signal, or a combined anti-noise, infrared signal to earphone speakers comprising:
an infrared transmitter and an infrared receiver therefore, said receiver coupled to first, second, and third circuits;
said first circuit being an anti-noise circuit comprising a microphone for detecting noise, phase detection circuit coupled to said microphone and to each speaker, and means for producing a second wave of opposite phase to said noise, coupled to said speaker;
volume control means coupled to said first circuit for manually controlling the volume of said second wave;
said second circuit comprising an infrared signal receiver and means for transmitting said signal from said receiver to said speakers;
second volume control means coupled between said receiver and speakers for controlling the volume of said signal transmitted;
said third circuit comprising power control means coupled between said infrared transmitter and said speakers for shutting off the power to said transmitter when a signal is not transmitted over a predetermined period of time; and
manual first and second switch means in, respectively, said first and second circuits, whereby control of said first and second switch means will permit either transmission of said infrared signal to said speakers with noise reduction, or transmission of the noise reduction signal only to said speakers.

2. The system of claim 1 wherein a first switch is coupled to said first circuit and a second switch is coupled to said second circuit, so that when both switches are closed, both circuits are operable to transmit a noise free signal, and when only said second switch is open, only the noise reduction signal will be transmitted.

3. The device of claim 1 further comprising an audio cable source selectively coupled to said second circuit whereby said circuit can receive an audio signal either from said infrared transmitter, or from said cable source.
